(19) United States
(12) Patent Application Publication
(10) Pub. No.: US 2026/0183038 A1
(43) Pub. Date: Jul. 2, 2026

(54) INTRALUMINAL ACCESSORY FOR ATRIO-SEPTAL SHUNT DILATION, CRYOBALLOON COUPLING, AND BLOOD SHIELDING

(71) Applicant: Medtronic, Inc., Minneapolis, MN (US)

(72) Inventors: Nicolas Coulombe, Anjou (CA); Mihai Stoianovici, Pierrefonds (CA); Ioana Deac, Vaudreuil-Dorion (CA)

(21) Appl. No.: 19/130,103

(22) PCT Filed: Nov. 8, 2023
(86) PCT No.: PCT/IB2023/061281

§ 371 Date: May 14, 2025

Related U.S. Application Data
(60) Provisional application No. 63/383,782, filed on Nov. 15, 2022.

(51) Int. Cl. A61B 18/02; A61B 18/00; A61B 18/14

(52) U.S. Cl. CPC A61B 18/02; A61B 18/1492; A61B 2018/00184; A61B 2018/00214; A61B 2018/00345; A61B 2018/00351; A61B 2018/00577; A61B 2018/00994; A61B 2018/0212

(57) ABSTRACT

An apparatus, system, and method for creating a shunt between a right atrium and a left atrium of a patient's heart. An apparatus includes a shielding structure, a tube proximal to the shielding structure, and an actuating mechanism at a proximal end of the tube. The tube is configured to pass through a lumen of a steerable catheter. The actuating mechanism is for deploying the shielding structure through the tube. The shielding structure is configured for passing through an opening in an atrial septum from the right atrium to the left atrium, and for being retracted in a proximal direction to contact the atrial septum for shielding a proximity of the opening from blood flow in the left atrium to facilitate cryoablation of the opening from the right atrium.

This application claims the benefit of U.S. Provisional Patent Application Ser. No. 63/383,782, filed Nov. 15, 2022, the entire content of which is incorporated herein by reference.

### TECHNICAL FIELD

The present technology is generally related to surgical devices, and more particularly, to devices and methods for creating an interatrial shunt.

### BACKGROUND

Atrial shunting is a surgical procedure used to treat certain cardiac defects and heart failure. During the procedure, a blood flow pathway, or shunt, is created between the right atrium and the left atrium of a patient's heart, such that blood flows between the right and left atria. In a typical procedure, the septal wall separating the atria is cut with a puncturing device and a mechanical device such as a stent is left in place to prevent tissue regrowth and to maintain the shunt. However, such procedures may result in tissue regrowth, thus reducing the effectiveness of the shunt. In other procedures, the tissue surrounding the septal wall may be ablated with thermal energy, such as cryogenic energy to prevent tissue regrowth and to maintain the shunt without the need for an implanted stent.

### SUMMARY

Around half of people suffering from heart failure also suffer from atrial fibrillation (AF). According to some aspects of this disclosure, an apparatus and method are provided that enable a surgeon to perform concomitant procedures for treating both heart failure and AF. For example, in some aspects, an accessory device is provided as an accessory to a common commercial cryoablation balloon used for a pulmonary vein isolation (PVI) procedure to treat AF. By the introduction of this accessory device, the same cryoablation balloon used for PVI can be used in a concomitant procedure for creating an implant-less interatrial shunt to treat for heart failure.

AF is a condition of abnormal or irregular beating of the atrial chambers of a patient's heart. One surgical treatment for AF is a PVI procedure. With PVI, a cardiologist ablates tissue in the left atrium to electrically isolate the left atrium from the pulmonary veins, where the abnormal electrical activity causing the AF may originate. PVI has become a common procedure. Commercial cryoablation balloons have been used in over a million patients.

Heart failure is a common syndrome in which a patient's heart output is insufficient to meet the body's needs. When a patient suffers from some forms of heart failure, the pressure in the left atrium may be higher than desired. To relieve the pressure in the left atrium, surgeons may use an implanted device such as a stent to create a shunt between the left and right atria. This interatrial shunting procedure can decompress the left atrium by creating a blood flow pathway between the right atrium and left atrium.

The techniques of this disclosure relate to procedures that create an interatrial shunt without the need for an implant. Such implant-less procedures eliminate the risk of implant failure and provide for continued access to the left atrium for future procedures. In some such implant-less procedures, a puncture or opening is created in the interatrial septum and the tissue surrounding the opening is ablated with thermal energy, such as cryogenic energy to prevent tissue regrowth and to maintain the shunt. However, in some cases, the cryogenic freezing may fail to extend through the full thickness of the atrial septum. For instance, a cryoablation balloon in the right atrium may only cause sufficient cryoablation in the right atrium side of the atrial septum. This is because the warm blood flow in the left atrium may continue to deliver heat to the left atrium side of the atrial septum, reducing the freezing effect. According to some aspects of this disclosure, a surgical apparatus and method is provided whereby a shielding structure in the left atrium shields the atrial wall from the warm blood flow, promoting a full thickness ablation from the right to the left atrium and creating a stable, implant-less interatrial shunt.

In one example, the disclosure describes an apparatus that includes a shielding structure, an elongate body proximal to the shielding structure, the elongate body configured to pass through a lumen of a steerable catheter, and an actuating mechanism at a proximal end of the elongate body for deploying the shielding structure via the elongate body. The shielding structure is configured for passing through an opening in an atrial septum of a heart of a patient from the right atrium to the left atrium, and for being retracted in a proximal direction to contact the atrial septum for shielding a proximity of the opening from blood flow in the left atrium to facilitate cryoablation of the opening from the right atrium.

In another example, the disclosure describes an apparatus including a steerable catheter comprising a lumen; a cryoablation balloon coupled near a distal end of the steerable catheter; an elongate body configured for passing through the lumen, the elongate body configured to pass through the cryoablation balloon; a shielding structure coupled to a distal end of the elongate body and configured to be positioned distal to the cryoablation balloon with the elongate body within the lumen; an actuating mechanism coupled to a proximal end of the elongate body for deploying the shielding structure; and a puncturing tool for forming an opening through an interatrial septum of a heart of a patient.

In another example, the disclosure describes a method including penetrating a septal wall between a right atrium and left atrium of a heart of a patient to create an opening; advancing a shielding structure through the opening into the left atrium; deploying the shielding structure in the left atrium to thermally shield the opening from blood in the left atrium; inflating a cryoablation balloon in the right atrium; applying tension between the cryoablation balloon and the shielding structure to bring the cryoablation balloon and the shielding structure into opposing contact with the septal wall; and applying a cryogenic fluid to the cryoablation balloon to ablate tissue of the septal wall proximate to the opening.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF THE DRAWINGS

FIG. 1 is a flow chart illustrating an example of a process for creating an interatrial shunt using an intraluminal accessory.

FIGS. 2-7 are a series of schematic illustrations of portions of the process of FIG. 1 as performed by an example surgical tool according to some aspects of this disclosure.

FIGS. 8A-8C are a series of schematic illustrations of portions of a process using an umbrella structure as a shielding structure according to some aspects of the disclosure.

FIGS. 9A-9B are illustrations of an accessory tool according to some aspects of the disclosure.

FIG. 10 is a flow chart illustrating another example of a process for creating an interatrial shunt using an intraluminal accessory.

### DETAILED DESCRIPTION

The disclosure describes examples of medical systems, devices, and techniques for creating a fluid pathway, or shunt, between the left atrium and right atrium of a heart of a patient without the use of an implant, e.g., such as a stent to maintain the shunt. The shunt may be formed in an atrial septum, and at least a portion of the shunt may be ablated. Example medical systems, devices, and techniques include a surgical tool including a steerable catheter and a cryoballoon, the steerable catheter including a lumen. The surgical tool may, in some examples, include a common commercial cryoballoon configured for a pulmonary vein isolation (PVI) procedure. An intraluminal accessory may engage with the surgical tool such that the surgical tool may also be used for creation of the interatrial shunt. The accessory may include a shielding structure, an elongate body (e.g., a tube) coupled to the shielding structure, and a controller or valve for deploying the shielding structure. The elongate body may be inserted into a lumen of the catheter, extending proximally from the shielding structure to the controller or valve at the proximal end of the catheter.

In accordance with example medical systems, devices, and techniques described herein, septal wall tissue may be ablated via cryoablation (e.g., via a cryogenic device such as a cryogenic balloon) to ablate a portion of the septal wall proximate to a puncture, opening, or shunt. Due to the nature of the ablation, the tissue adjacent to the ablation will fibrose/endothelialize and define an opening (e.g., a shunt) that may be formed between the left atrium and the right atrium, enabling pressure from the left atrium to decompress into the right atrium. This may help treat heart failure, such as by mitigating a mechanism of heart failure. In other examples, the systems, devices, and techniques described herein can be used to create a shunt between two other hollow anatomical structures of a patient and to treat other patient conditions. Thus, while a shunt between a left atrium and a right atrium of a heart of a patient is primarily referred to herein, the systems, devices, and techniques can be used to form shunts in other locations of the heart, other locations of the body of patients, or for other medical procedures in other examples.

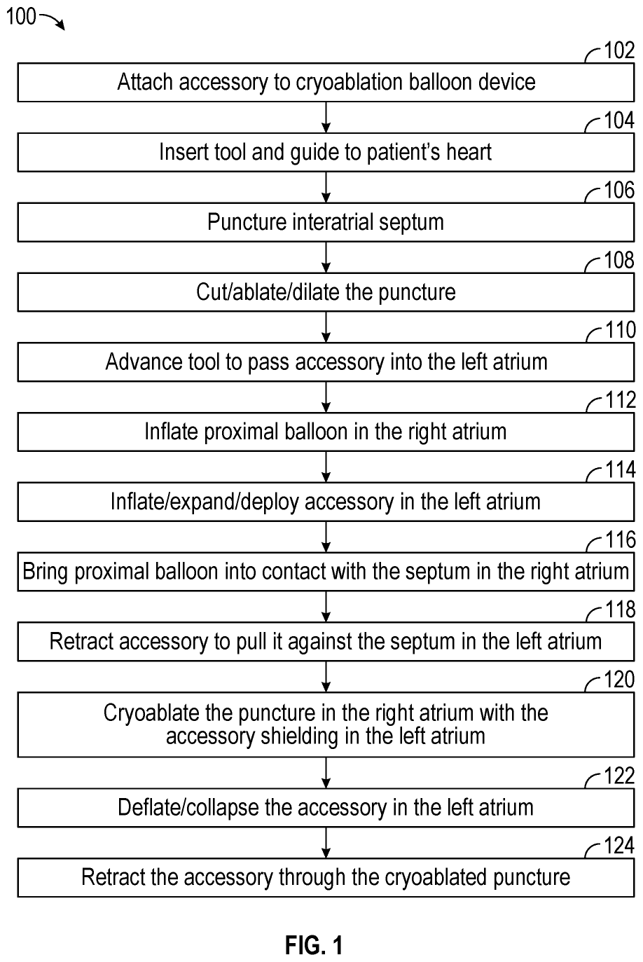

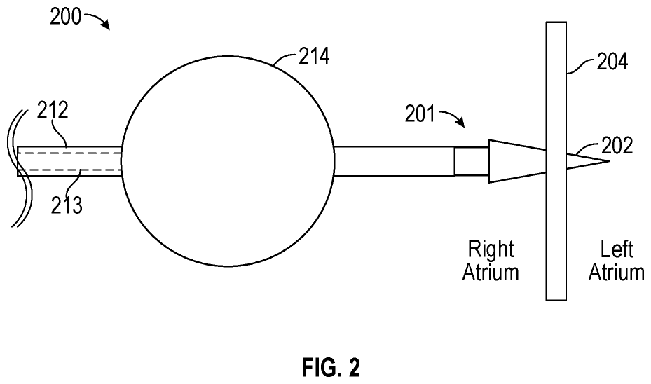

FIG. 1 is a flow chart illustrating an exemplary process 100 for creating an interatrial shunt according to some aspects of this disclosure. The description below of the process 100 includes reference to FIGS. 2-7, which schematically illustrate a portion of a procedure for creating an interatrial shunt. The example technique of FIGS. 1-7 is described with reference to a surgical tool 200 and an accessory device 201, however, the example technique may be performed using any system including a device and/or tool including the functionality of the accessory device 201 described herein. The technique of FIGS. 1-7 may be performed by any suitable user, such as a cardiologist or other clinician.

In the examples illustrated by FIGS. 2-7, surgical tool 200 includes a catheter 212, a cryoablation balloon 214 at or near a distal end of the surgical tool 200, and a lumen 213 passing through the catheter 212. In some examples, the catheter 212 may be a steerable catheter, configured for guiding through a patient's circulatory system. In other examples, the lumen 213 may include a guidewire lumen, such that the surgical tool 200 may be guided through a patient's circulatory system via a guidewire. In some examples, the surgical tool 200 may be a common commercial cryoablation tool configured for pulmonary vein isolation (PVI) treatment for atrial fibrillation (AF). The proximal end of the surgical tool 200 may be configured for attachment to a suitable valve or controller for delivering air, a cryogenic fluid, and/or any other suitable fluid to the cryoablation balloon. The controller may further include suitable controls or mechanisms for steering the tool to the patient's heart and controlling a surgical procedure.

According to an aspect of this disclosure, an accessory device 201 may be attached to or otherwise engaged with the surgical tool 200 (102). For example, the accessory device 201 may be inserted into the lumen 213 at a proximal end of the surgical tool 200. In an implementation where the accessory 201 is inserted into the lumen 213 at the proximal end of the surgical tool 200, a balloon or shielding structure of the accessory may be configured to have a small size when deflated or collapsed, to fit through the lumen 213 to pass in a distal direction across the cryoablation balloon of the surgical tool 200. However, depending on the nature of the accessory 201, and its shielding structure or balloon, it may be too large to pass fully through the lumen 213. Thus, in another example, the accessory device 201 may be attached to the distal end of the surgical tool 200, e.g., being inserted into a lumen 213 at the distal end of the surgical tool 200, distal to the cryoablation balloon 214. Here, the accessory device 201 may include a wire or tube that extends in the proximal direction from the distal end of the surgical tool 200 across the cryoablation balloon 214 to the proximal end of the surgical tool 200. In some examples, the accessory device 201 may be attached to the surgical tool 200 via a coupler (not illustrated) that is secured to the lumen's luer and fastens to the shaft of the accessory device 201.

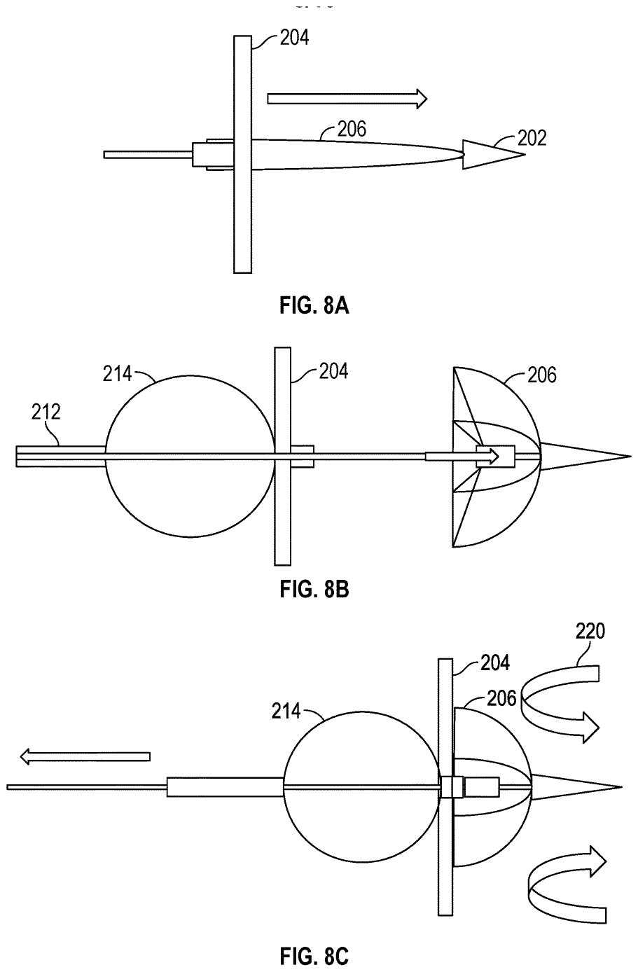

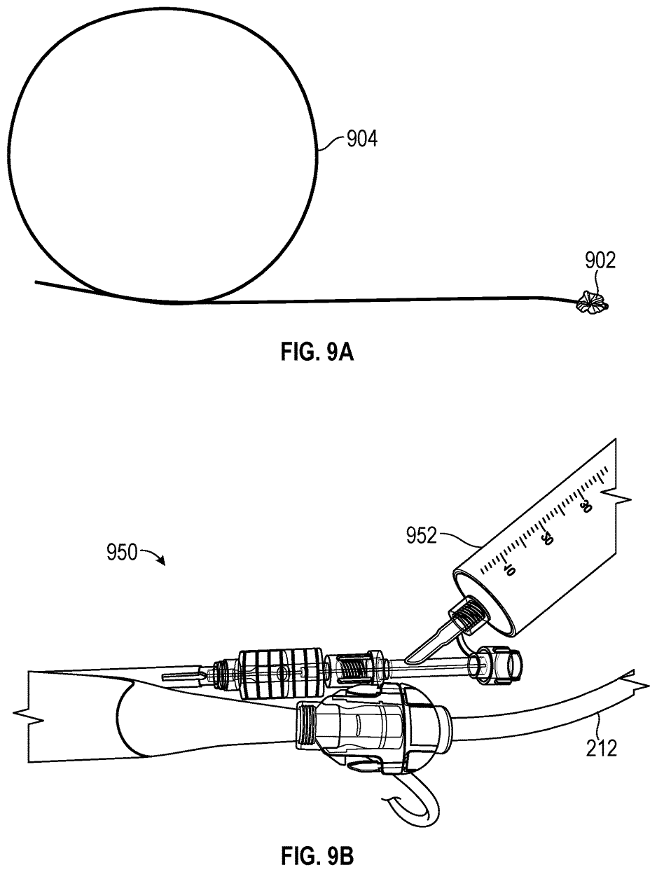

The accessory device 201 and/or the surgical tool 200 may further include a suitable actuating mechanism (e.g., a valve or controller) configured to be attached at the proximal end of the surgical tool 200. For example, when the accessory device 201 includes a balloon 208, a valve (e.g., see FIG. 9B) may be attached at the proximal end of the surgical tool 200 such that air can pass through a tube passing through the lumen 213 of the catheter 212 to inflate the balloon 208. In various examples, the balloon 208 of the accessory device 201 may be inflated independently from the cryoablation balloon 214. In another example, where the accessory device 201 includes a mechanically expandable shielding structure (e.g., see FIG. 8 below), the actuating mechanism may be a valve or controller attached at the proximal end of the surgical tool 200 for pneumatically or mechanically controlling deployment (e.g., expansion and collapse) of the shielding structure.

The surgical tool 200, with attached accessory 201, may be inserted (104) into a suitable blood vessel in the patient, such as the femoral vein, and guided to the patient's heart. A clinician may steer the surgical tool using a steerable sheath such that the surgical tool enters the right atrium of the patient's heart, e.g., via the inferior vena cava.

Once the surgical tool is in the right atrium of the patient's heart, a puncturing tool 202 may be used to puncture (106) the interatrial septum 204 of the patient's heart. FIG. 2 is a schematic illustration of this step in a surgical procedure, showing a surgical tool in the right atrium of a patient's heart. The surgical tool 200 includes a puncturing tool 202 for puncturing through the interatrial septum 204 into the left atrium of the patient's heart. Any suitable puncturing tool 202 may be used, including but not limited to a needle, a knife, an electrical ablation tool, etc. Puncturing tool 202 may be included as part of the accessory 201 as shown in FIG. 2, or may be a separate tool, e.g., separately delivered through the lumen of surgical tool 200 or otherwise delivered to interatrial septum 204. While FIG. 2 shows a proximal cryoablation balloon 214 in an inflated state, this is merely for illustrative purposes and cryoablation balloon 214 need not necessarily be inflated at this phase of the procedure.

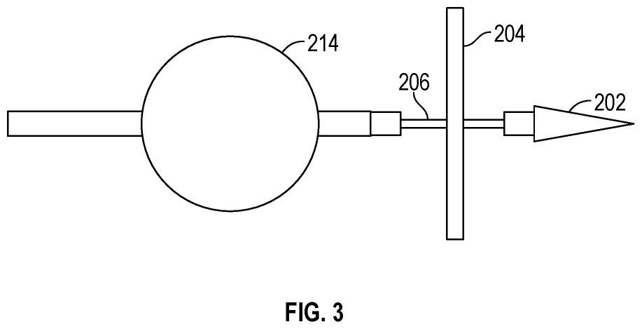

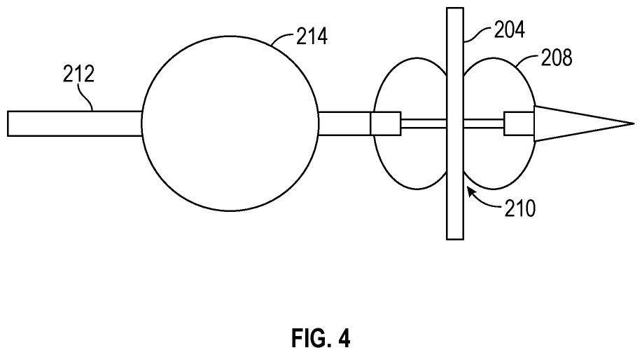

FIG. 3 shows the puncturing tool 202 extending into the left atrium of the patient's heart. In the illustration of FIG. 3, a shielding structure 206 has passed about halfway through the atrial septum 204. In some aspects, after puncturing the atrial septum 204, the surgical tool may cut, ablate, or dilate the puncture (108) to increase the size of the puncture opening. For example, FIG. 4 shows an example wherein the shielding structure 206 is an inflatable balloon 208. In some examples, the inflatable balloon 208 may be a two-lobed balloon, with a waist 210 between a pair of lobes of the balloon 208. In some examples, the waist 210 may be sized and shaped to create a controlled dilation of the puncture, expanding the size of the puncture to a controlled diameter. The two lobes of the balloon 208 may serve to center the balloon 208 in the puncture, ensuring that the diameter of the waist 210 is the diameter of the dilated opening. In some other examples, the inflatable balloon 208 may be a single-lobe balloon having a diameter that, when forced through the puncture in the atrial septum 204, dilates the puncture to a controlled diameter. In a further aspect, the shielding structure 206 may be constructed of a mesh-like material (e.g., metallic alloy or a suitable polymer material) that is covered with a balloon. Here, a mesh can provide increased rigidity for a more controlled stretching or dilation of the puncture opening. In any case, the balloon 208 may be inflated via a tube (not illustrated) configured to pass through the catheter 212 across the cryoablation balloon 214 to an actuating mechanism such as an inflating valve located at a proximal end of the catheter 212.

In other examples within the scope of this disclosure, any suitable means or mechanism for cutting, ablating, or dilating the puncture to increase its size may be used, not being limited to a balloon structure. For example, a metallic accessory such as a plasma blade using RF energy may be used to ablate or cut the opening.

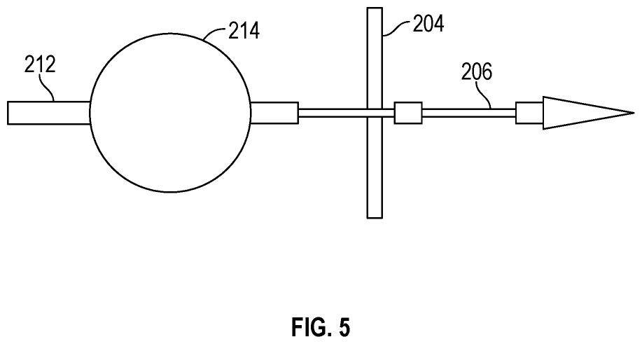

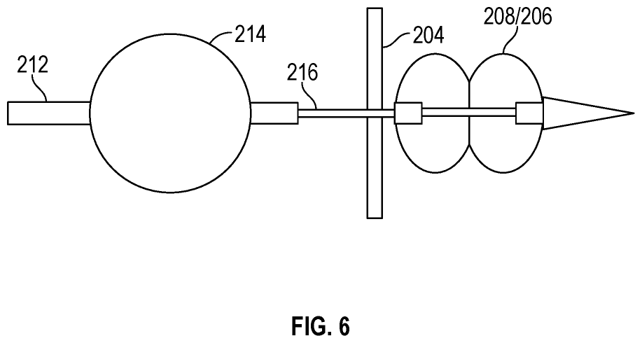

In an example where the balloon 208 is used to dilate the puncture opening, once the opening is dilated, as illustrated in FIG. 5, the balloon may be deflated and the shielding structure 206 may be advanced (110) to pass the shielding structure 206 through the atrial septum 204 and into the left atrium. When the shielding structure 206 is in the left atrium, or at any other suitable time, the proximal cryoablation balloon 214 may be inflated (112) in the right atrium of the patient's heart, using air or any other suitable fluid. As illustrated in FIG. 6, the shielding structure 206 (e.g., the distal balloon 208) in the left atrium may be re-inflated, expanded, or deployed (114), e.g., by inflating the distal balloon 208 using air or any other suitable fluid. Thus, as shown in FIG. 6, shielding structure 206 (e.g., distal balloon 208) may be inflated, expanded, or deployed in a left atrium of a patient's heart while a proximal balloon 214 (i.e., a cryoablation balloon) may be inflated in a right atrium of the patient's heart, with a portion 216 of the catheter 212 in between, passing through the opening in the atrial septum 204.

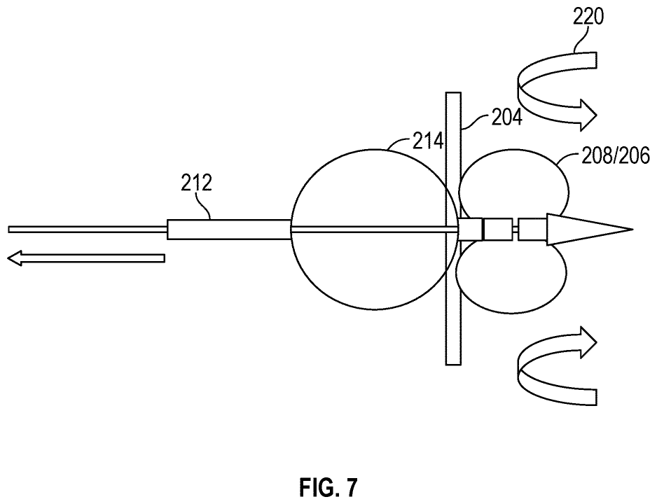

In a further aspect, as illustrated in FIG. 7, the proximal balloon (e.g., cryoablation balloon 214) may be brought into contact (116) with the atrial septum 204 in the right atrium of the patient's heart. For example, the catheter 212 may be used to advance the cryoablation balloon 214 toward the opening in the atrial septum 204. Further, the distal shielding structure 206 (e.g., the distal balloon 208) may be retracted (118) to contact the atrial septum 204 in the left atrium of the patient's heart. For example, the portion 216 of the catheter 212 (see FIG. 6) may be retracted through the cryoablation balloon 214 to apply a tension between the proximal cryoablation balloon 214 and the distal shielding structure 206, thus reducing the distance between the proximal cryoablation balloon 214 and the distal shielding structure 206 (e.g., the distal balloon 208). In this manner, the atrial septum 204 may be sandwiched between the proximal cryoablation balloon 214 and the distal shielding structure 206.

In an aspect of this disclosure, the shielding structure 206 may be a balloon 208 filled with a thermally insulating fluid, such as air, and constructed of a material that is impervious to blood. Any suitable blood-impervious material may be used to construct the balloon 208, including a polymer material such as polyurethane, nylon, PET, or other suitable thermoplastic, or a mesh composed of a polymer such as nylon. When the balloon 208 is retracted against the septum 204 in the proximity of the puncture opening, the shielding structure 206 shields the septum 204 from blood and, particularly from a blood heat load in the left atrium. In this manner, when the cryoablation balloon 214 is filled with a suitable cryogenic fluid to cryoablate (120) the puncture in the right atrium, the shielding structure 206 (e.g., the distal balloon 208) thermally insulates the puncture in the left atrium from warm blood 220 circulating in the left atrium. Thus, a cryoablation effect is more likely to occur through the full thickness of the atrial septum 204 where the cryoablation balloon 212 is in contact with the atrial septum 204. Furthermore, because the cryoablation balloon 212 and the distal shielding structure 206 are in tension with one another with the puncture opening in the atrial septum 204 in between, the cryoablation balloon 212 is more likely to be centered within the puncture opening, creating an even cryoablation effect around the puncture opening.

In the illustration of FIG. 7, the distal shielding structure 206 is a balloon structure 208. In an aspect of this disclosure, when the catheter 212 is retracted to pull the balloon 208 into the atrial septum 204, the balloon 208 may take the general shape of a donut or torus. FIG. 7 shows this torus-like shape in cross section. Of course, it is not necessary for a shielding structure 206 to take a torus-like shape when shielding the atrial septum 204. In other examples, a balloon may remain generally spherical or may take any other suitable shape for shielding the atrial septum 204. Moreover, in other examples described in this disclosure, the shielding structure may be an umbrella-like structure or some other suitable structure for shielding the atrial septum 204 from blood flow 220 in the left atrium.

When the cryoablation procedure is complete, the distal shielding structure 206 may be deflated or collapsed (122) in the left atrium, and retracted (124) through the cryoablated puncture into the right atrium. The cryoablation balloon 212 may also be deflated, and in some examples, retracted into the catheter 212, and the surgical tool may be removed from the patient.

However, in a further aspect of the disclosure, when the cryoablation procedure is complete, the distal shielding structure 206 may be deflated or collapsed in the left atrium, and the cryoablation balloon 212 may also be deflated or collapsed in the right atrium. The cryoablation balloon 212 may then be advanced through the cryoablated puncture into the right atrium. From this point, the cryoablation balloon 212 may be further used for a conventional PVI procedure in the left atrium, wherein the pulmonary veins are electrically isolated from the heart by ablating tissue in the left atrium. The details of a PVI procedure are not provided in the present disclosure as a person having ordinary skill in the relevant art would be familiar with a PVI procedure.

In yet another aspect of the disclosure, a PVI procedure may be performed (with or without the accessory 201 engaged with the surgical tool 200) before the creation of the interatrial shunt. That is, in various aspects, by using the apparatus and methods disclosed herein, an interatrial shunt procedure may be performed before or after performing a PVI procedure utilizing the cryoablation balloon and accessory as described herein.

FIGS. 8A-8C illustrate a further aspect of the disclosure, wherein the shielding structure 206 has an umbrella-like structure. The shielding structure 206 may be part of an accessory device 201 as described above, engaged with a surgical tool including a puncturing tool 202 and a catheter 212. The umbrella-like structure may be any suitable collapsible structure constructed of any suitable material, including a metallic alloy such as Nitinol, a polymer material such as nylon, or polyester. In FIG. 8A, the umbrella-like structure is collapsed as the shielding structure 206 passes through the atrial septum 204 from the right atrium to the left atrium. In some examples, the collapsed umbrella-like structure may be used to dilate the opening in the atrial septum 204, although this is not necessarily the case. In other examples, the umbrella-like structure may not be used to dilate the opening. In some further examples, the metallic alloy forming the umbrella-like structure may be energized to function as electrodes for radio frequency ablation of tissue of the atrial septum 204. And in further examples, the umbrella-like structure may include one or more electrodes (not illustrated) on an outer surface, for radio frequency ablation of tissue of the atrial septum 204. In this manner, the umbrella-like structure in a collapsed or partially collapsed configuration may be used for cutting the interatrial septum 204 in a controlled manner as the shielding structure 206 is passed through the interatrial septum 204 from the right atrium to the left atrium.

In FIG. 8B, the shielding structure 206 is fully deployed in the left atrium. As discussed above, the shielding structure 206 may be mechanically or pneumatically coupled to an actuating mechanism at a proximal end of the surgical tool. Thus, a surgeon may deploy the shielding structure using the actuating mechanism, causing the umbrella-like structure to expand. In FIG. 8B, the umbrella-like structure in its expanded configuration has a generally hemispherical shape, although this is not necessarily the case. The umbrella-like structure in its expanded configuration may have an oval shape, a conic shape, a bell shape, or any other suitable shape for shielding the interatrial septum 204 from blood in the left atrium.

In FIG. 8C, the shielding structure 206 is retracted, sandwiching the atrial septum 204 between the cryoablation balloon 214 and the shielding structure 206. Thus, the shielding structure 206 thermally isolates the area of the atrial septum 204 in the proximity of the puncture opening from blood flow 220 in the left atrium.

FIGS. 9A and 9B illustrate one example of an accessory device 900 according to some aspects of this disclosure. In the illustration of FIG. 9A, the accessory device 900 is withdrawn from a surgical tool 200 (not illustrated). The accessory device 900 includes a shielding structure 902, and an elongate body 904 coupled to the shielding structure 902. The elongate body 904 may be a tube or other suitable structure for facilitating inflation or deployment of the shielding structure 902. In the illustrated example, the shielding structure 902 is a balloon in a deflated state, although as described above, different shielding structures may be used within the scope of this disclosure. The elongate body 904 may be configured to pass through a lumen of a steerable catheter. As described above, the elongate body 904 may include a lumen coupled to the shielding structure 902 for deploying the shielding structure. For example, the elongate body 904 may be configured to deliver air or other suitable fluid to a balloon when the shielding structure 902 is a balloon. FIG. 9B illustrates a portion of a controller 950 at a proximal end of a catheter 212. As described above, the elongate body 904 may be configured to extend from a distal tip of the surgical tool, through a lumen, to a proximal end of the catheter 212 where the tube may be coupled to a controller or valve 952 for deploying the shielding structure 902. For example, when the shielding structure 902 is a balloon, a syringe 952 may be used to controllably inflate the balloon.

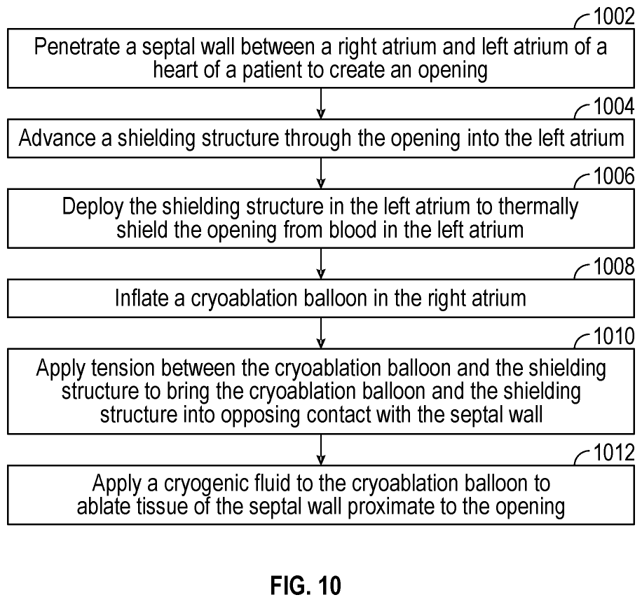

FIG. 10 is a flow chart illustrating an exemplary process 1000 of creating an interatrial shunt according to some aspects of this disclosure. The techniques of FIG. 10 may be performed by any suitable user, such as a cardiologist or other clinician.

Inside the right atrium of a patient's heart, a surgical tool may penetrate (1002) a septal wall between the right atrium and a left atrium of the heart to create an opening. For example, a distal tip of the surgical tool may include a needle, knife, or radio frequency ablation tool for penetrating the septal wall. The surgical tool may then advance (1004) a shielding structure through the opening into the left atrium. For example, a catheter may be used to advance a shielding structure such as a balloon or umbrella-like structure through the opening. In some examples, the shielding structure may be used to dilate the opening as it is advanced through the opening. For example, a balloon may expand within the opening to dilate the opening. In another example, the shielding structure may include an electrode for radio frequency ablation of the opening as the shielding structure is advanced through the opening.

When fully in the left atrium, the shielding structure may be deployed (1006) to thermally shield the opening from blood in the left atrium. For example, when the shielding structure is a balloon, the balloon may be inflated in the left atrium and retracted to contact the atrial septum in the proximity of the opening. In another example, when the shielding structure is an umbrella-like structure, the umbrella may be mechanically or pneumatically expanded in the left atrium and retracted to contact the atrial septum similar to the balloon.

A cryoablation balloon that remains in the right atrium may be inflated (1008) using any suitable fluid, including but not limited to air. A tension may be applied (1010) between the cryoablation balloon and the shielding structure to bring the cryoablation balloon and the shielding structure into opposing contact with the septal wall. For example, a portion of the catheter may be retracted to apply a tension between the cryoablation balloon and the shielding structure. A cryogenic fluid may then be applied (1012) to the cryoablation balloon to ablate tissue of the septal wall proximate to the opening. For example, a cryogenic fluid may be deployed inside the cryoablation balloon where the cryoablation balloon contacts the septal wall. In this way, the tissue of the septal wall proximate to the opening may be ablated to create a stable shunt without need for an implant, such as a stent.

Accordingly, although example systems and techniques have been shown and described, it is to be understood that all the terms used herein are descriptive rather than limiting, and that many changes, modifications, and substitutions may be made by one having ordinary skill in the art without departing from the spirit and scope of the invention. The following examples are examples of systems, devices, and methods described herein.

Example 1: In some examples, a surgical apparatus includes a shielding structure; an elongate body proximal to the shielding structure, the elongate body configured to pass through a lumen of a steerable catheter; and an actuating mechanism at a proximal end of the elongate body for deploying the shielding structure via the elongate body. The shielding structure is configured for passing through an opening in an atrial septum of a heart of a patient from the right atrium to the left atrium, and for being retracted in a proximal direction to contact the atrial septum for shielding a proximity of the opening from blood flow in the left atrium to facilitate cryoablation of the opening from the right atrium.

Example 2: In some examples of the surgical apparatus of Example 1, the shielding structure includes a balloon. Deploying the shielding structure includes pressurizing the actuating mechanism to inflate the balloon through the elongate body.

Example 3: In some examples of the surgical apparatus of Examples 1 to 2, the shielding structure is further configured for dilating the opening in the atrial septum to a controlled diameter as the shielding structure passes through the opening.

Example 4: In some examples of the surgical apparatus of Examples 1 to 3, the shielding structure includes a dual-lobe balloon comprising a waist having the controlled diameter for dilating the opening in the atrial septum.

Example 5: In some examples of the surgical apparatus of Example 1, the shielding structure includes an umbrella-like structure. The umbrella-like structure is configured for being in a collapsed configuration when passing through the opening in the atrial septum. Deploying the shielding structure includes using the actuating mechanism to expand the umbrella-like structure pneumatically or mechanically via the elongate body.

Example 6: In some examples of the surgical apparatus of Examples 1 or 5, the umbrella-like structure further includes an electrode configured for radio frequency ablation of the atrial septum.

Example 7: In some examples, a surgical system includes a steerable catheter including a lumen; a cryoablation balloon coupled near a distal end of the steerable catheter; an elongate body configured for passing through the lumen, the elongate body configured to pass through the cryoablation balloon; a shielding structure coupled to a distal end of the elongate body and configured to be positioned distal to the cryoablation balloon with the elongate body within the lumen; an actuating mechanism coupled to a proximal end of the elongate body for deploying the shielding structure; and a puncturing tool for forming an opening through an interatrial septum of a heart of a patient.

Example 8: In some examples of the surgical system of Example 7, the shielding structure includes a balloon. Deploying the shielding structure includes pressurizing the actuating mechanism to inflate the balloon through the elongate body.

Example 9: In some examples of the surgical system of Examples 7 to 8, the shielding structure is configured for passing through the opening in the atrial septum from the right atrium to the left atrium, and for being retracted in a proximal direction to thermally insulate a portion of the atrial septum proximate to the opening from blood flow in the left atrium to facilitate cryoablation of the opening from the right atrium by the cryoablation balloon.

Example 10: In some examples of the surgical system of Examples 7 to 9, the shielding structure is further configured for dilating the opening in the atrial septum to a controlled diameter as the shielding structure passes through the opening.

Example 11: In some examples of the surgical system of Examples 7 to 10, the shielding structure includes a dual-lobe balloon comprising a waist having the controlled diameter for dilating the opening in the atrial septum.

Example 12: In some examples of the surgical system of Examples 7, 9, or 10, the shielding structure includes an umbrella-like structure. The umbrella-like structure is configured for being in a collapsed configuration when passing through the opening in the atrial septum. Deploying the shielding structure includes using the actuating mechanism to expand the umbrella-like structure pneumatically or mechanically via the elongate body.

Example 13: In some examples of the surgical system of Examples 7, 9, 10, or 12, the umbrella-like structure further includes an electrode configured for radio frequency ablation of the atrial septum.

Example 14: In some examples of the surgical system of Examples 7 to 13, the cryoablation balloon is further configured for a pulmonary vein isolation (PVI) procedure in the left atrium.

Example 15: In some examples, a surgical method includes penetrating a septal wall between a right atrium and left atrium of a heart of a patient to create an opening; advancing a shielding structure through the opening into the left atrium; deploying the shielding structure in the left atrium to thermally shield the opening from blood in the left atrium; inflating a cryoablation balloon in the right atrium; applying tension between the cryoablation balloon and the shielding structure to bring the cryoablation balloon and the shielding structure into opposing contact with the septal wall; and applying a cryogenic fluid to the cryoablation balloon to ablate tissue of the septal wall proximate to the opening.

Example 16: In some examples of the surgical method of Example 15, advancing the shielding structure through the opening includes dilating the opening with the shielding structure.

Example 17: In some examples of the surgical method of Examples 15 to 16, the shielding structure includes a balloon. Dilating the opening includes inflating the balloon within the opening. Deploying the shielding structure includes inflating the balloon within the left atrium.

Example 18: In some examples of the surgical method of Examples 15 to 16, the shielding structure includes an umbrella-like structure having an electrode on an outer surface. Dilating the opening includes applying radio frequency ablation via the electrode.

Example 19: In some examples of the surgical method of Examples 15 to 18, the method further includes advancing the cryoablation balloon through the opening into the left atrium; and ablating tissue in the left atrium to electrically isolate a pulmonary vein from the left atrium.

The techniques described in this disclosure may be implemented, at least in part, in hardware, software, firmware or any combination thereof. For example, various aspects of the described techniques may be implemented within one or more processors or processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

1. An apparatus comprising:
a shielding structure;
an elongate body proximal to the shielding structure, the elongate body configured to pass through a lumen of a steerable catheter; and
an actuating mechanism at a proximal end of the elongate body for deploying the shielding structure via the elongate body,
wherein the shielding structure is configured for passing through an opening in an atrial septum of a heart of a patient from the right atrium to the left atrium, and for being retracted in a proximal direction to contact the atrial septum for shielding a proximity of the opening from blood flow in the left atrium to facilitate cryoablation of the opening from the right atrium.

2. The apparatus of claim 1, wherein the shielding structure comprises a balloon, and wherein deploying the shielding structure comprises pressurizing the actuating mechanism to inflate the balloon through the elongate body.

3. The apparatus of claim 1, wherein the shielding structure is further configured for dilating the opening in the atrial septum to a controlled diameter as the shielding structure passes through the opening.

4. The apparatus of claim 1, wherein the shielding structure comprises a dual-lobe balloon comprising a waist having the controlled diameter for dilating the opening in the atrial septum.

5. The apparatus of claim 1, wherein the shielding structure comprises an umbrella-like structure,
wherein the umbrella-like structure is configured for being in a collapsed configuration when passing through the opening in the atrial septum, and
wherein deploying the shielding structure comprises using the actuating mechanism to expand the umbrella-like structure pneumatically or mechanically via the elongate body.

6. The apparatus of claim 5, wherein the umbrella-like structure further comprises an electrode configured for radio frequency ablation of the atrial septum.

7-8. (canceled)

9. A system comprising:
a steerable catheter comprising a lumen;
a cryoablation balloon coupled near a distal end of the steerable catheter;
an elongate body configured for passing through the lumen, the elongate body configured to pass through the cryoablation balloon;
a shielding structure coupled to a distal end of the elongate body and configured to be positioned distal to the cryoablation balloon with the elongate body within the lumen; an actuating mechanism coupled to a proximal end of the elongate body for deploying the shielding structure; and
a puncturing tool for forming an opening through an interatrial septum of a heart of a patient.

10. The system of claim 9, wherein the shielding structure comprises a balloon, and wherein deploying the shielding structure comprises pressurizing the actuating mechanism to inflate the balloon through the elongate body.

11. The system of claim 9, wherein the shielding structure is configured for passing through the opening in the atrial septum from the right atrium to the left atrium, and for being retracted in a proximal direction to shield a portion of the atrial septum proximate to the opening from blood flow in the left atrium to facilitate cryoablation of the opening from the right atrium by the cryoablation balloon.

12. The system of any claim 9, wherein the shielding structure is further configured for dilating the opening in the atrial septum to a controlled diameter as the shielding structure passes through the opening.

13. The system of claim 9, wherein the shielding structure comprises a dual-lobe balloon comprising a waist having the controlled diameter for dilating the opening in the atrial septum.

14. The system of claim 9, wherein the shielding structure comprises an umbrella-like structure,
wherein the umbrella-like structure is configured for being in a collapsed configuration when passing through the opening in the atrial septum, and
wherein deploying the shielding structure comprises using the actuating mechanism to expand the umbrella-like structure pneumatically or mechanically via the elongate body.

15. The system of claim 14, wherein the umbrella-like structure further comprises an electrode configured for radio frequency ablation of the atrial septum.

16. The system of claim 9, wherein the cryoablation balloon is further configured for a pulmonary vein isolation (PVI) procedure in the left atrium.

17. A method comprising:
penetrating a septal wall between a right atrium and left atrium of a heart of a patient to create an opening;
advancing a shielding structure through the opening into the left atrium; deploying the shielding structure in the left atrium to shield the opening from blood in the left atrium;
inflating a cryoablation balloon in the right atrium;
applying tension between the cryoablation balloon and the shielding structure to bring the cryoablation balloon and the shielding structure into opposing contact with the septal wall; and
applying a cryogenic fluid to the cryoablation balloon to ablate tissue of the septal wall proximate to the opening.

18. The method of claim 17, wherein advancing the shielding structure through the opening comprises:
dilating the opening with the shielding structure.

19. The method of either of claims 17, wherein the shielding structure comprises a balloon,
wherein dilating the opening comprises inflating the balloon within the opening, and
wherein deploying the shielding structure comprises inflating the balloon within the left atrium.

20. The method of either of claims 17, wherein the shielding structure comprises an umbrella-like structure having an electrode on an outer surface, and wherein dilating the opening comprises applying radio frequency ablation via the electrode.

21. The method of claim 17, further comprising:
advancing the cryoablation balloon through the opening into the left atrium; and
ablating tissue in the left atrium to electrically isolate a pulmonary vein from the left atrium.
